(19) United States
(12) Patent Application Publication
(10) Pub. No.: US 2025/0259019 A1
(43) Pub. Date: Aug. 14, 2025

(54) ROUTER-GUIDED KNOWLEDGE INFUSION

(71) Applicant: NEC Laboratories America, Inc., Princeton, NJ (US)

(72) Inventors: Runxue Bao, Redmond, WA (US); Yanchi Liu, Princeton, NJ (US); Wei Cheng, Princeton Junction, NJ (US); Wenchao Yu, Plainsboro, NJ (US); Haifeng Chen, West Windsor, NJ (US)

(21) Appl. No.: 19/051,847

(22) Filed: Feb. 12, 2025

Related U.S. Application Data
(60) Provisional application No. 63/552,393, filed on Feb. 12, 2024.

(51) Int. Cl. G06F 40/40; G06N 3/042; G06N 3/0455; G16H 20/00

(52) U.S. Cl. CPC G06F 40/40; G06N 3/042; G06N 3/0455; G16H 20/00

(57) ABSTRACT

Methods and systems include determining that a query is relevant to information that is unknown to a pre-trained language model. Outputs from adapter layers are added to outputs of respective transformer layers of the language model to infuse the language model with the information, such that the language model generates a response to the query that accounts for the information that is unknown to the pre-trained language model. An action is performed based on the response.

### RELATED APPLICATION INFORMATION

This application claims priority to U.S. Patent Application No. 63/552,393, filed on Feb. 12, 2024, incorporated herein by reference in its entirety.

### BACKGROUND

### Technical Field

The present invention relates to large language models and, more particularly, to enhancing large language models with knowledge graphs.

### Description of the Related Art

Large language models (LLMs) are trained on a large corpus of unsupervised text and can perform well on a variety of natural language processing tasks. LLMs may undergo a two-stage training process. An initial self-supervised pre-training stage is performed on large, unlabeled datasets. A second task-specific fine-tuning stage may be performed on smaller, labeled datasets. However, despite their adaptability to a broad range of language tasks, the performance of LLMs in knowledge-intensive scenarios may be hampered by knowledge gaps.

### SUMMARY

A method includes determining that a query is relevant to information that is unknown to a pre-trained language model. Outputs from adapter layers are added to outputs of respective transformer layers of the language model to infuse the language model with the information, such that the language model generates a response to the query that accounts for the information that is unknown to the pre-trained language model. An action is performed based on the response.

A system includes a hardware processor and a memory that stores a computer program. When executed by the hardware processor, the computer program causes the hardware processor to determine that a query is relevant to information that is unknown to a pre-trained language model, to add outputs from adapter layers to outputs of respective transformer layers of the language model to infuse the language model with the information, such that the language model generates a response to the query that accounts for the information that is unknown to the pre-trained language model, and to perform an action based on the response.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of a method for identifying information that is unknown to a pre-trained large language model (LLM), in accordance with an embodiment of the present invention;

FIG. 2 is a block diagram of a knowledge infusion system that uses an adapter to supplement the information encoded in an LLM with information from a knowledge graph, in accordance with an embodiment of the present invention;

FIG. 3 is a block diagram of an adapter that supplements information in an LLM using a router to selectively incorporate that information, in accordance with an embodiment of the present invention;

FIG. 4 is a block/flow diagram of a method of training and using an adapter that infuses domain-specific information into the output of a pre-trained LLM, in accordance with an embodiment of the present invention;

FIG. 5 is a diagram of a vehicle with an automated driving system that can use knowledge infusion to supplement a control system using a pre-trained LLM, in accordance with an embodiment of the present invention;

FIG. 6 is a diagram of a healthcare facility that can use an LLM with knowledge infusion to guide medical decision making using specialized medical knowledge bases, in accordance with an embodiment of the present invention;

FIG. 7 is a block diagram of a computing device that can infuse information from a knowledge graph into the output of an LLM, in accordance with an embodiment of the present invention;

FIG. 8 is a diagram of an exemplary neural network architecture that can be used to implement parts of a router model, in accordance with an embodiment of the present invention; and

FIG. 9 is a diagram of an exemplary deep neural network architecture that can be used to implement parts of a router model, in accordance with an embodiment of the present invention.

### DETAILED DESCRIPTION OF PREFERRED EMBODIMENTS

Large language models (LLMs) may be enhanced by the integration of domain-specific knowledge from knowledge graphs. Knowledge graphs are characterized by their structured, quantifiable, and dynamically updatable nature and can serve as a reservoir for domain-specific knowledge. However, integration of knowledge graph-derived information into an LLM can be computationally expensive if it is integrated in the pre-training or fine-tuning stages.

A parameter-efficient, adapter-based framework may therefore be used to fine-tune an LLM to help integrate the information of a knowledge graph. Router-guided knowledge infusion is used to help prevent catastrophic forgetting, where the integration of new knowledge might otherwise erase previously acquired information. This makes it possible to make strategic decisions regarding the integration of new knowledge with existing knowledge, ensuring that the infusion process enriches the LLM without compromising its existing knowledge base. The result is that the LLM is augmented with domain-specific knowledge, thereby enhancing its applicability and performance in knowledge-intensive natural language processing tasks.

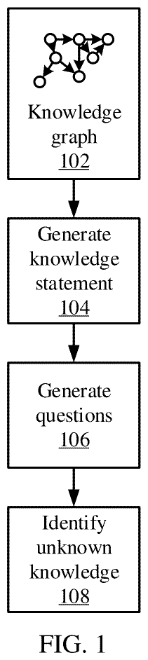

Referring now to FIG. 1, an illustration of knowledge detection is shown. A knowledge infusion framework detects the LLM's knowledge boundaries using sampled knowledge sub-graphs 102. For each knowledge subject-predicate-object triplet <h, r, t>, where h stands for head, r stands for relation, and t stands for tail, the router-guided knowledge infusion framework generates a knowledge statement 104 and multiple-choice questions 106 that employ predefined relational templates. Based on the answers to these test questions, block 108 identifies unknown knowledge from the knowledge graph 102.

Knowledge graphs 102 are used as knowledge sources due to their comprehensive and quantifiable nature, which offers advantages over textual documents. This helps LLMs by providing a complete knowledge body and defining their knowledge boundaries. To evaluate the initial knowledge boundaries of LLMs, the knowledge of the knowledge graphs, expressed as triples, is converted to the multiple-choice questions 106.

A pretrained LLM may be used to transform the knowledge triples into knowledge statements 104 and questions 106. For example, a triplet of <Sutura cranii, has finding site, Acrocephalosyndactyly type 5>may be rephrased into the question, “What diagnosis is associated with the finding site of Sutura cranii?” and the answer, “Acrocephalosyndactyly type 5.” The knowledge statement may be, “The finding site for Sutura cranii is associated with Acrocephalosyndactyly type 5.”

The multiple-choice format may be used to enable a more effective comparison of the long-form answers produced by LLMs with standard answers in open-ended questions. Each question in this format includes the correct answer and three distractors. One of the distractors is selected based on its closest edit distance to the head entity. The other two may be sampled from a pool of potential distractors by their edit distance to the answer entity. These options may then be shuffled and presented in an (A)(B)(C)(D) format with the question, enabling precise evaluation of the LLM's proficiency in domain-specific knowledge graphs.

After creating the multiple-choice questions, they are input to the LLM with a testing prompt. LLMs may provide long answers, so regular expressions may be used to extract the chosen options, treating the response as incorrect if no options can be extracted. This helps to identify the LLM's knowledge boundary, indicating what it knows and what it does not. The set of unknown knowledge may be represented as G_{U}.

A routing mechanism can use parallel adapters to encode new knowledge into the LLM while preserving the original parameters of the model. The LLM's internal state can be used to reveal the truthfulness of sentences that the LLM itself generates. By evaluating the LLM's truthfulness through its internal states, the routing mechanism selectively provides supplementary information for unknown topics 108, thereby minimizing inference for previously acquired information. Relationships may be classified to improve the linguistic representations learned by the adapter, predicting relations within knowledge statements based on the adapter outputs on head and tail entities. This solidifies the integration of new knowledge and helps the robustness of generalizing the new knowledge to unseen situations.

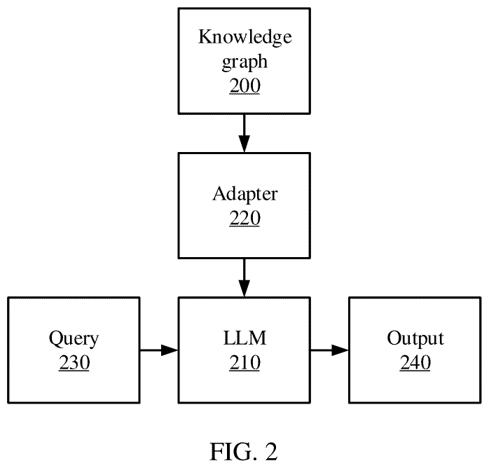

Referring now to FIG. 2, a diagram of knowledge infusion is shown. An LLM 210 is integrated with an external knowledge graph 200 through a knowledge adapter 220. The adapter 220 integrates knowledge-driven multiple-choice question-answering 106 and knowledge statements 104, thereby enhancing the proficiency of the LLM 210 in specialized domains. During runtime, a query 230 is processed by the adapter to determine whether to use information from the knowledge graph 200. If so, the information from the knowledge graph 200 is added to the query 230 and is processed by the LLM 210 to generate an output 240. In instances where the specialized information of the knowledge graph 200 is not needed, then the LLM 210 processes the query 230 using only its own pretrained parameters.

Formally, given an LLM p_{θ}∈ [symbol] and a set of knowledge triples [symbol] ∈ [symbol] , the adapter 220 aims to fine-tune the model to p_{θ}′ to add unknown knowledge without affecting existing knowledge. For efficiency, only information that is unknown to the LLM ( [symbol] ₙₖ) is used, skipping known information [symbol] _{known}. Thus:

KGI: [symbol] × [symbol] → [symbol] p_{θ}′=KGI(p_{θ}, [symbol] ᵤₙₖ)

where KGI stands for Knowledge Graph Infusion. Domain-specific knowledge triplets are transformed into multiple-choice questions. The models' responses to these questions serve to assess the LLM's comprehension of the relevant knowledge triple. Upon establishing an unknown knowledge question-answer set, the knowledge adapter 220 effectively integrates the unknown set using multiple-choice question-answering and relationship classification tasks, along with a routing mechanism for information interaction with the LLM 210. Distinct loss functions, including a QA loss, a routing loss, a next token prediction loss, and a relationship classification loss, may be used to ensure reliability, locality, and generality throughout the knowledge infusion process.

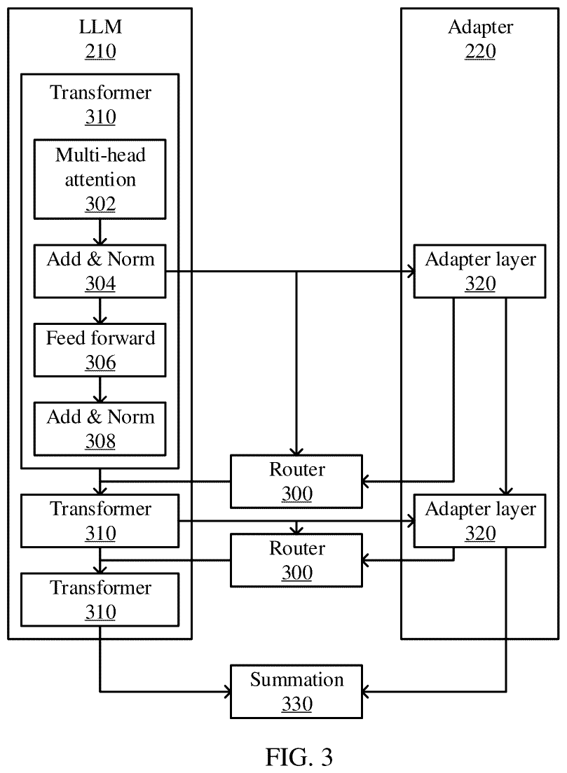

Referring now to FIG. 3, additional detail on the adapter 220 is shown along with its integration with the LLM 210. The LLM 210 is made up of a series of transformer layers 310, each of which may include a multi-head attention 302, a first Add & Norm operation 304, a feed forward operation 306, and a second Add & Norm operation 308. The output of the first Add & Norm layer 304 is provided as inputs to a corresponding adapter layer 320 of the adapter and to a corresponding router 300.

Based on the output of the first Add & Norm layer 304, the router makes a determination of whether to introduce information from the adapter layer 320. If so, then that information is combined with the output of the transformer layer 310 before it is used as the input of the next transformer layer. The output of the adapter layer 320 is used as input to a subsequent adapter layer 320. Once the LLM 210 has generated an output, that output may be combined with an output of the final adapter layer 320 of the adapter 220 by summation 330.

To improve parameter efficiency, parallel adapters may be used as extra models, keeping the original LLM parameters unchanged. Feed forward layers store knowledge effectively, so the adapter layers 320 are added in parallel. For each adapter layer l of a set of L adapter layers (l∈[1, L]), the transformer's input H_{P}^{l}∈ [symbol] ^{n×d} is combined with the output H_{A}^{l-1} from the previous adapter layer:

$${\tilde{H}}_{A}^{l} = {H}_{A}^{l - 1} + {H}_{P}^{l}$$

Here n is the length of the LLM input sequence and d is a hidden size.

First the adapter layer 320 projects the dimension of the combined input H_{A}^{l} down to a low dimension, then another linear projection layer is applied to project it up to its original dimension d:

H_{A}^{l}=UP(ST(DOWN(\tilde{H}_{A}^{l})))

The UP function is represented by a d′×d matrix. A non-linear activation function, such as a sigmoid linear unit function, is applied immediately after the UP function. The DOWN function is represented by a d×d′ matrix.

The router 300 filters the adapter output, avoiding the addition of unnecessary information. The router 300 may be implemented as a multi-layer perceptron (MLP). An output routing value of the router 300 may be determined as:

r^{l}=Router(Mean(H_{P}^{l}))

Where the Mean function averages the vector along the sequence length. Finally, an additive filtered adapter vector is generated, which is integrated with the original transformer output:

$${H}_{O}^{l} = {r}^{l} ⊗ {H}_{A}^{l} + FFN ( {H}_{P}^{l} )$$

To enhance the efficiency of integrating knowledge into LLMs from both data and training perspectives, unfamiliar knowledge is used exclusively through conditional text generation. Parallel adapter layers 320 are used to store this newly acquired knowledge. The unknown knowledge is identified in the knowledge detection stage. Specifically, question-based instructions are used with standard answers as golden responses. A QA loss may be tailored to adapt instructions within a specified domain:

$${ℒ}_{QA} = {𝔼}_{x , y} [ \frac{1}{\vert y \vert} {\sum }_{i = 1}^{\vert y \vert} CE ( {P}_{θ} ( \cdot \vert x , {y}_{1 , … , i - 1} ) , {y}_{i} ) ]$$

where CE denotes a cross-entropy loss function, yᵢ is a golden output, and P_{θ} is a prediction output of an LLM. A small set of yes/no question-answering samples may be used to enhance the model generality to various question types.

A routing mechanism assesses whether the LLM 210 is familiar with the knowledge at hand. If not, the adapter 220 steps in to provide extra information. If they are already familiar with the knowledge, then the adapter 220 has less impact. A routing value r^{l}∈[0,1] is added during the integration of the adapter and original outputs. This value, created by router 300, judges how well the LLM 210 knows the current knowledge, based on intermediate representations in the l-th Transformer layer (H_{P}^{l}). However, the router 300 only sees new knowledge and might not recognize old knowledge well. To fix this, some samples of knowledge the LLM already have are introduced. Before fine-tuning, the Router is pre-trained on a binary routing task with a balanced mix of familiar and unfamiliar samples. The Router loss is a binary cross-entropy loss function:

[symbol]

_{Router}= [symbol] _{x,y}_{_{Router}}[BCE(f_{Router}(H_{P}^{l}),y_{Router})]

Here, f_{Router} calculates a routing score from 0 to 1 on how much the LLM 210 knows about the knowledge. The routing label y_{Router} is 1 for new knowledge and 0 for familiar knowledge. This mechanism helps LLMs learn new things without forgetting what they already know.

Knowledge graphs uniquely represent knowledge in a precise triple format, offering a distinct advantage. Capitalizing on this feature, this task is dedicated to improving adapters' understanding of relational facts for more effective use of this representation. Given the entity-related knowledge statement k and its corresponding triple [symbol] h, r, t [symbol] , a mean pooling operation is applied over the embeddings from the adapter output H_{A}^{L} of the entity mentions to obtain entity representations v^{h} and v^{t}. These are concatenated as the relational representation v^{r}=[v^{h}, v^{t}]. The relation value r is a positive sample and other relations are regarded as negatives to better understand relational facts. The Relation Classification (RC) loss function is:

$${ℒ}_{RC} = {𝔼}_{k} [ - log \frac{exp ( {f}_{1}^{R} ( {v}^{r} ) \cdot {f}_{2}^{R} ( r ) / τ )}{{\sum }_{{r}^{′} ∈ ε} exp ( {f}_{1}^{R} ( {v}^{r} ) \cdot {f}_{2}^{R} ( {r}^{′} ) / τ )} ]$$

Here, τ is a temperature hyper-parameter, k is a knowledge statement with triple <h, r, t>, v^{h} and v^{t} are mean-pooled adapter outputs H_{A}^{L} for entity mentions, f₂^{R}(r) is the positive sample, similarly pooled from the LLM embedding layer, with other relations as negatives, f₁^{R} projects head and tail embeddings to align dimensionally with the relation representations, and ε is the set of relations.

Besides that, a next token loss is used:

$${ℒ}_{NTL} = {𝔼}_{k} [ \frac{1}{\vert k \vert} {\sum }_{i = 1}^{\vert k \vert} CE ( {P}_{θ} ( {k}_{i} \vert {k}_{1 , … , i - 1} ) ) ]$$

Given a question-based instruction q, a standard response y, a knowledge statement k, and a knowledge triple [symbol] h, r, t [symbol] , the training is divided into three stages. In the initial stage, the router 300 is pre-trained using a small set of balanced samples of information that the LLM already knows and information that it does not already know. In the second stage, the model is fine-tuned using the QA loss to integrate unknown knowledge. In the final stage, knowledge statements and triples are used to enhance model generalization. Overall the training objective of the adapter 220 is:

$$ℒ = {ℒ}_{Router} + {ℒ}_{QA} + {ℒ}_{NTL} + {ℒ}_{RC}$$

Optimizing this objective function using the knowledge from the knowledge graph causes that knowledge to be encoded in the parameters of the adapter 220.

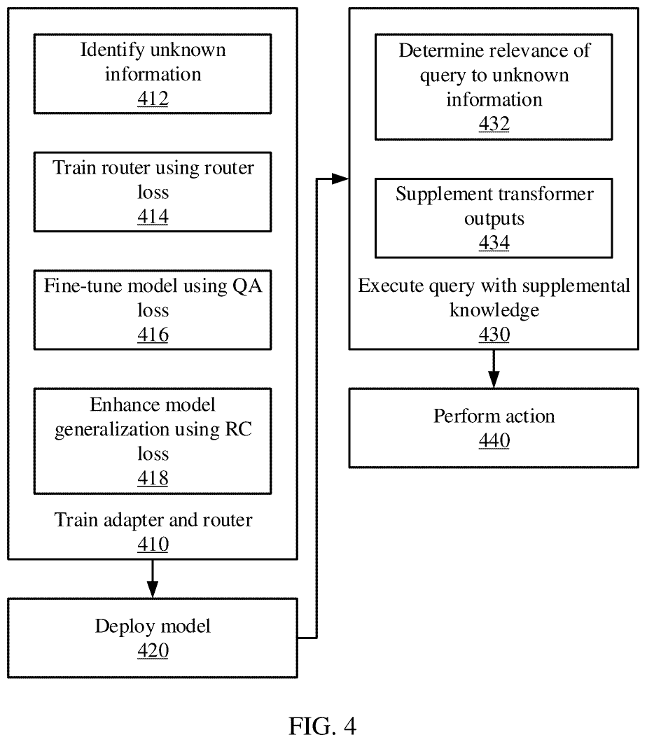

Referring now to FIG. 4, a method of training and using a knowledge infusion adapter is shown. Block 410 trains the adapter, which includes training the router 300 and adapter layers 320. Notably the parameters of the transformer layers 310 are pre-trained and fixed during this process. This training includes identifying 412 information that the LLM 210 does not know. Block 414 trains the router 300 using the router loss [symbol] _{Router}, block 416 fine-tunes the model, including the router 300 and the adapter layers 320, using the QA loss [symbol] _{QA}, and block 418 enhances model generalization using the RC loss [symbol] _{RC}.

Block 420 deploys the trained adapter model. In some cases, where the adapter model is trained at the same system which will perform later inferences, the deployment step may be omitted. In other cases, the trained parameters of the router 300 and the adapter layers 320 may be transmitted to another system to be implemented in conjunction with the LLM 210.

Block 430 executes a query using supplemental knowledge from the knowledge base. Block 432 determines the relevance of the query to information that the LLM 210 does not possess, but which is encoded in the knowledge graph. Block 434 then supplements the transformer outputs using information from the adapter 220, with the router 300 acting to combine the information on a layer-by-layer basis. The adapter 220 has information from the knowledge graph encoded within its parameters, and so adding its outputs to the outputs of transformers 310 incorporates that information into the LLM's output.

Block 440 performs an action responsive to the output of the LLM 210. In particular, the query may be directed to specialized, domain-specific information, and the output of the LLM 210 will reflect that domain-specific information from the knowledge graph.

Thus the action 440 may include simple operation of a chat bot, where questions relating to domain-specific information can be answered with greater accuracy than the LLM would be able to do alone. In some cases the output of the LLM may be integrated with an automated system, for example using the query to provide instructions to a system that uses domain-specific information to perform an automated response.

As an example of such a system, block 440 may perform an action in an automated driving system. The query may instruct the automated driving system to navigate to a particular location, where the location and route is defined by the knowledge graph. As a further example, block 440 may perform an automated treatment action when the query asks for a diagnosis of a patient based on the patient's medical condition. The domain-specific knowledge of the knowledge graph may include medical information that was not used to train the LLM.

For example, this action may be implemented in the context of an automated driving system, which receives a query from a user or from another system component. The query may instruct the automated driving system to navigate to a particular destination, where the specific location, route details, and related contextual information, such as real-time traffic data or road closure information, are derived from the domain-specific knowledge graph.

In this example, the knowledge graph may incorporate specialized mapping data, regional regulations, and weather conditions that were not present in the training corpus of the LLM 210. By using these additional data sources, the automated driving system can optimize route planning, improve safety by anticipating hazardous road conditions, and dynamically adjust navigation decisions when new information is received. The action 440 may thus include a braking action, a steering action, and/or an acceleration action.

As another example, the action may be an automated treatment action in a healthcare setting. When a query is made regarding a patient's diagnosis, taking into account symptoms, medical history, and other clinical indicators, the knowledge graph can provide domain-specific details that extend beyond the LLM's general medical knowledge. For example, the knowledge graph may include specialized protocols for rare diseases, localized treatment guidelines for specific patient populations, or updated research findings that were released after the LLM's original training period. By fusing these curated, frequently updated data sets with the LLM's language understanding capabilities, the system can support more accurate diagnoses, suggest personalized treatment options, and streamline clinical decision making. This dual-source approach ensures that medical providers and automated systems alike benefit from robust natural language processing and the latest context-specific information, resulting in more effective and informed actions across diverse application domains.

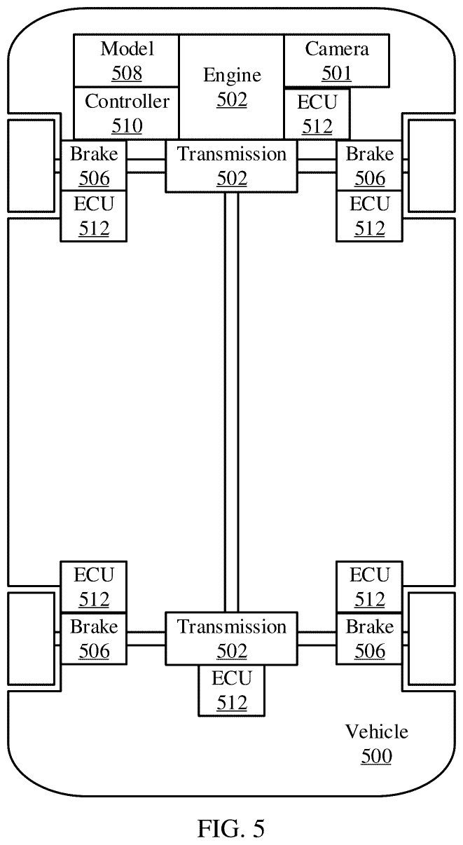

Referring now to FIG. 5, a vehicle 500 is shown. A number of different sub-systems of the vehicle 500 are shown, including an engine 502, a transmission 504, and brakes 506. It should be understood that these sub-systems are provided for the sake of illustration, and should not be interpreted as limiting. Additional sub-systems may include user-facing systems, such as climate control, user interface, steering control, and braking control. Additional sub-systems may include systems that the user does not directly interact with, such as tire pressure monitoring, location sensing, collision detection and avoidance, and self-driving.

Each sub-system is controlled by one or more equipment control units (ECUs) 512, which perform measurements of the state of the respective sub-system. For example, ECUs 512 relating to the brakes 506 may control an amount of pressure that is applied by the brakes 506. An ECU 512 associated with the wheels may further control the direction of the wheels. The information that is gathered by the ECUs 512 is supplied to the controller 510. A camera 501 or other sensor (e.g., LiDAR or RADAR) can be used to collect information about the surrounding road scene, and such information may also be supplied to the controller 510.

Communications between ECUs 512 and the sub-systems of the vehicle 500 may be conveyed by any appropriate wired or wireless communications medium and protocol. For example, a car area network (CAN) may be used for communication. The time series information may be communicated from the ECUs 512 to the controller 510, and instructions from the controller 510 may be communicated to the respective sub-systems of the vehicle 500.

Information from the camera 501 and other sensors is provided to the model 508, which may select an appropriate action to take. The controller 510 uses the output of the model 508, based on information collected from cameras 501, to perform a driving action responsive to the present state of the scene. The controller 510 may make use of an LLM 210 with an adapter 220 that is trained to incorporate specialized knowledge as described above.

The controller 510 may communicate internally to the sub-systems of the vehicle 500 and the ECUs 512. Based on detected road fault information, the controller 510 may communicate instructions to the ECUs 512 to avoid a hazardous road condition. For example, the controller 510 may automatically trigger the brakes 506 to slow down the vehicle 500 and may furthermore provide steering information to the wheels to cause the vehicle 500 to move around a hazard.

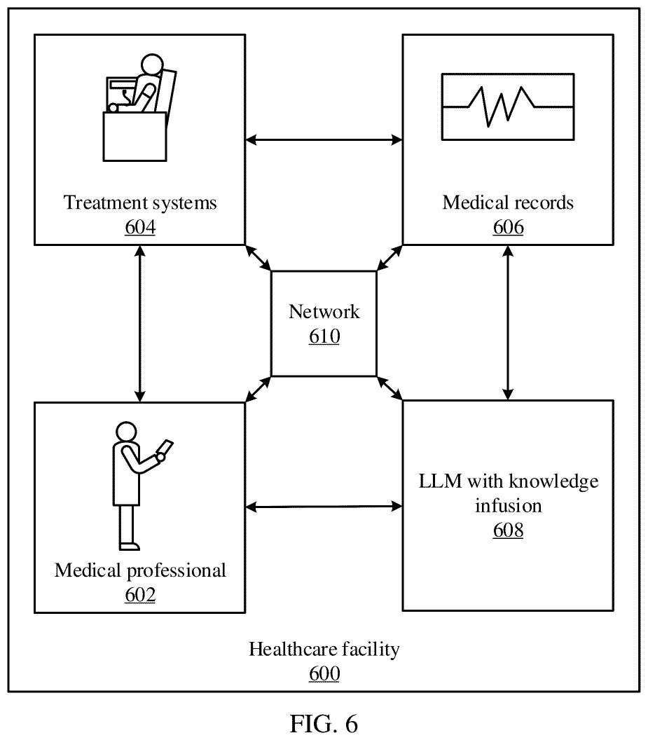

Referring now to FIG. 6, a diagram of knowledge infusion is shown in the context of a healthcare facility 600. An LLM with knowledge diffusion 608 may be used to guide medical decision making by incorporating information from specialized knowledge bases, for example performing diagnoses using specialized information that is deemed relevant to a patient's condition. The LLM with knowledge diffusion 608 may be used to generate treatment recommendations relating to a patient's medical condition based on up-to-date medical records 606.

The healthcare facility may include one or more medical professionals 602 who review information extracted from a patient's medical records 606 to determine their healthcare and treatment needs. These medical records 606 may include self-reported information from the patient, test results, and notes by healthcare personnel made to the patient's file. Treatment systems 604 may furthermore monitor patient status to generate medical records 606 and may be designed to automatically administer and adjust treatments as needed.

Based on information provided by the LLM with knowledge diffusion 608, the medical professionals 602 may make medical decisions about patient healthcare suited to the patient's needs. For example, the medical professionals 602 may make a diagnosis of the patient's health condition and may prescribe particular medications, surgeries, and/or therapies.

The different elements of the healthcare facility 600 may communicate with one another via a network 610, for example using any appropriate wired or wireless communications protocol and medium. Thus the LLM with knowledge diffusion 608 can be used to design a treatment that targets a patient's specific condition, for example using tissue samples and medical records 606. The treatment systems 604 may be used to generate and administer a therapy based on the output of the LLM with knowledge diffusion 608.

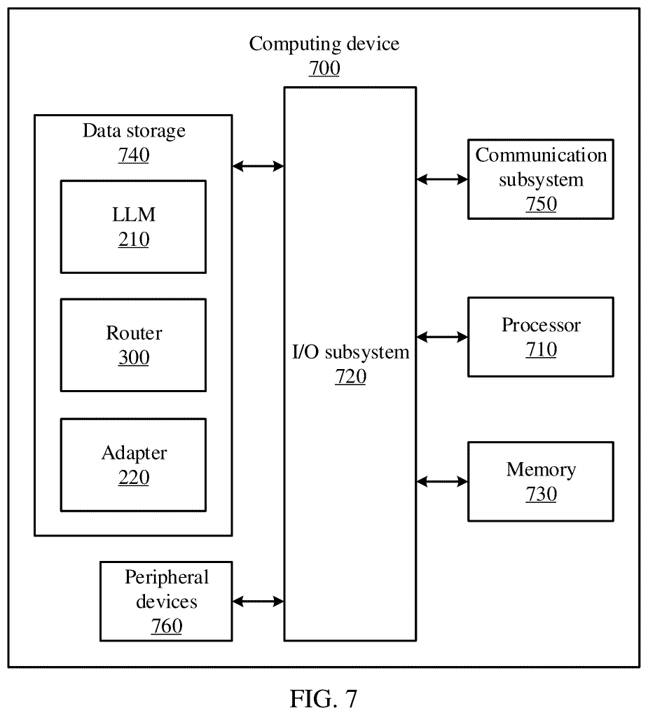

As shown in FIG. 7, the computing device 700 illustratively includes the processor 710, an input/output subsystem 720, a memory 730, a data storage device 740, and a communication subsystem 750, and/or other components and devices commonly found in a server or similar computing device. The computing device 700 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 730, or portions thereof, may be incorporated in the processor 710 in some embodiments.

The processor 710 may be embodied as any type of processor capable of performing the functions described herein. The processor 710 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 730 may be embodied as any type of volatile or non-volatile memory or data storage capable of performing the functions described herein. In operation, the memory 730 may store various data and software used during operation of the computing device 700, such as operating systems, applications, programs, libraries, and drivers. The memory 730 is communicatively coupled to the processor 710 via the I/O subsystem 720, which may be embodied as circuitry and/or components to facilitate input/output operations with the processor 710, the memory 730, and other components of the computing device 700. For example, the I/O subsystem 720 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 720 may form a portion of a system-on-a-chip (SOC) and be incorporated, along with the processor 710, the memory 730, and other components of the computing device 700, on a single integrated circuit chip.

The data storage device 740 may be embodied as any type of device or devices configured for short-term or long-term storage of data such as, for example, memory devices and circuits, memory cards, hard disk drives, solid state drives, or other data storage devices. The data storage device 740 can store program code 740A for an LLM, 740B, for a router 300, and/or 740C for an adapter that infuses specialized information from a knowledge base into the LLM's output. Any or all of these program code blocks may be included in a given computing system. The communication subsystem 750 of the computing device 700 may be embodied as any network interface controller or other communication circuit, device, or collection thereof, capable of enabling communications between the computing device 700 and other remote devices over a network. The communication subsystem 750 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 700 may also include one or more peripheral devices 760. The peripheral devices 760 may include any number of additional input/output devices, interface devices, and/or other peripheral devices. For example, in some embodiments, the peripheral devices 760 may include a display, touch screen, graphics circuitry, keyboard, mouse, speaker system, microphone, network interface, and/or other input/output devices, interface devices, and/or peripheral devices.

Of course, the computing device 700 may also include other elements (not shown), as readily contemplated by one of skill in the art, as well as omit certain elements. For example, various other sensors, input devices, and/or output devices can be included in computing device 700, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 700 are readily contemplated by one of ordinary skill in the art given the teachings of the present invention provided herein.

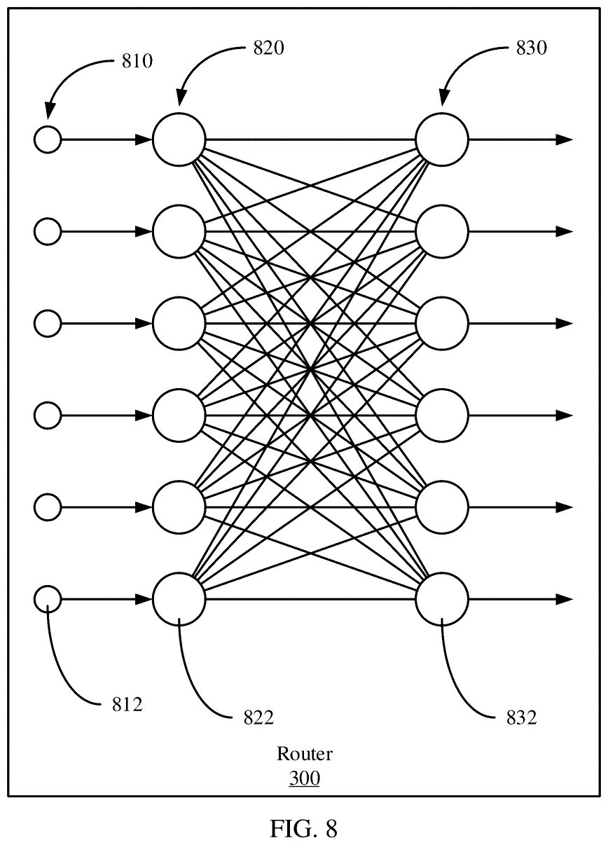

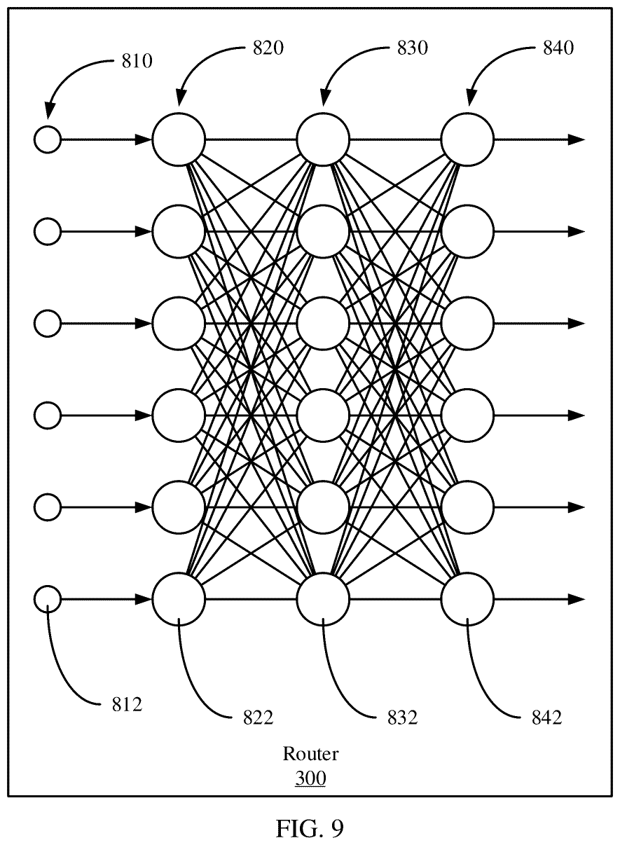

Referring now to FIGS. 8 and 9, exemplary neural network architectures are shown, which may be used to implement parts of the present models, such as the router 300. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 820 of source nodes 822, and a single computation layer 830 having one or more computation nodes 832 that also act as output nodes, where there is a single computation node 832 for each possible category into which the input example could be classified. An input layer 820 can have a number of source nodes 822 equal to the number of data values 812 in the input data 810. The data values 812 in the input data 810 can be represented as a column vector. Each computation node 832 in the computation layer 830 generates a linear combination of weighted values from the input data 810 fed into input nodes 820, and applies a non-linear activation function that is differentiable to the sum. The exemplary simple neural network can perform classification on linearly separable examples (e.g., patterns).

A deep neural network, such as a multilayer perceptron, can have an input layer 820 of source nodes 822, one or more computation layer(s) 830 having one or more computation nodes 832, and an output layer 840, where there is a single output node 842 for each possible category into which the input example could be classified. An input layer 820 can have a number of source nodes 822 equal to the number of data values 812 in the input data 810. The computation nodes 832 in the computation layer(s) 830 can also be referred to as hidden layers, because they are between the source nodes 822 and output node(s) 842 and are not directly observed. Each node 832, 842 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 832 in the one or more computation (hidden) layer(s) 830 perform a nonlinear transformation on the input data 812 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
determining that a query is relevant to information that is unknown to a pre-trained language model;
adding outputs from adapter layers to outputs of respective transformer layers of the language model to infuse the language model with the information, such that the language model generates a response to the query that accounts for the information that is unknown to the pre-trained language model; and
performing an action based on the response.

2. The method of claim 1, wherein determining that the query is relevant uses a router neural network and wherein adding the outputs is performed by the router neural network.

3. The method of claim 2, further comprising training the router and the adapter layers using a knowledge graph that includes the information.

4. The method of claim 3, wherein training the router and the adapter includes minimizing a loss function:
$$ℒ = {ℒ}_{Router} + {ℒ}_{QA} + {ℒ}_{NTL} + {ℒ}_{RC}$$
where [symbol] _{Router} is a binary cross-entropy loss, [symbol] _{QA} is a question-answer loss that adapts instructions within a specified domain, [symbol] _{NTL} is a next-token loss, and [symbol] _{RC} is a relation classification loss.

5. The method of claim 3, wherein training the router includes generating questions and knowledge statements relating to the information.

6. The method of claim 1, wherein each adapter layer receives information from a respective transformer layer and adds its output to the output of the respective transformer layer.

7. The method of claim 1, wherein parameters of the adapter layers encode the information.

8. The method of claim 1, wherein the action includes performing an action in an automated driving system, selected from the group consisting of a braking action, a steering action, and an acceleration action.

9. The method of claim 1, wherein the action includes performing a treatment action responsive to the query being based on a patient's medical condition.

10. The method of claim 1, wherein the information includes domain-specific information that was not used during training of the pre-trained language model.

11. A system, comprising:
a hardware processor; and
a memory that stores a computer program which, when executed by the hardware processor, causes the hardware processor to:
determine that a query is relevant to information that is unknown to a pre-trained language model;
add outputs from adapter layers to outputs of respective transformer layers of the language model to infuse the language model with the information, such that the language model generates a response to the query that accounts for the information that is unknown to the pre-trained language model; and
perform an action based on the response.

12. The system of claim 11, wherein the computer program causes the hardware processor to a router neural network to determine that the query is relevant and wherein addition of the outputs is performed by the router neural network.

13. The system of claim 12, wherein the computer program further causes the hardware processor to train the router and the adapter layers using a knowledge graph that includes the information.

14. The system of claim 13, wherein the training of the router and the adapter includes minimizing a loss function:
$$ℒ = {ℒ}_{Router} + {ℒ}_{QA} + {ℒ}_{NTL} + {ℒ}_{RC}$$
where [symbol] _{Router} is a binary cross-entropy loss, [symbol] _{QA} is a question-answer loss that adapts instructions within a specified domain, [symbol] _{NTL} is a next-token loss, and [symbol] _{RC} is a relation classification loss.

15. The system of claim 13, wherein the training of the router includes generating questions and knowledge statements relating to the information.

16. The system of claim 11, wherein each adapter layer receives information from a respective transformer layer and adds its output to the output of the respective transformer layer.

17. The system of claim 11, wherein parameters of the adapter layers encode the information.

18. The system of claim 11, wherein the action includes an action in an automated driving system, selected from the group consisting of a braking action, a steering action, and an acceleration action.

19. The system of claim 11, wherein the action includes a treatment action responsive to the query being based on a patient's medical condition.

20. The system of claim 11, wherein the information includes domain-specific information that was not used during training of the pre-trained language model.
